(19) United States
(12) Patent Application Publication
(10) Pub. No.: US 2013/0014866 A1
(43) Pub. Date: Jan. 17, 2013

(54) METHOD FOR RECLAIMING HIGH EXPLOSIVE FROM WARHEAD BY MELTING-OUT IN SUPERCRITICAL FLUID

(71) Applicants: JIN-SHUH LI, Fenyuan (TW); Taso-Fa Yeh, Fenyuan (TW); Kai-Tai Lu, Fenyuan (TW); Yung-Ho Chiu, Fenyuan (TW); Shuo-Hsiu Chen, Fenyuan (TW)

(72) Inventors: JIN-SHUH LI, Fenyuan (TW); Taso-Fa Yeh, Fenyuan (TW); Kai-Tai Lu, Fenyuan (TW); Yung-Ho Chiu, Fenyuan (TW); Shuo-Hsiu Chen, Fenyuan (TW)

(21) Appl. No.: 13/182,583

(22) Filed: Jul. 14, 2011

(51) Int. Cl. C06B 21/00; C07C 201/16

(52) U.S. Cl. 149/109.6; 568/935

(57) ABSTRACT

A method for the retraction of an explosive component from a high explosive, including the steps of loading a high explosive containing an explosive component into an extraction vessel. A supercritical fluid is supplied to the extraction vessel. The high explosive is contacting with the supercritical fluid at a temperature below the melting point of the explosive component and at a pressure sufficient to extract the explosive component.

### BACKGROUND OF THE INVENTION

1. Field of the Invention

The invention relates to methods for recovery and separation of high explosive from aged munitions and more particularly to a method for reclaiming a high explosive from warhead by melting-out in supercritical fluid.

2. Description of Related Art

How to effectively remove obsolete and aged munitions and explosive inventories from the active arsenal in a safe manner is of great concern. Typically, demilitarization programs focused on disposal or destruction. Recently, there have been efforts to develop recycling and/or reclamation processes that permit explosives and higher valued constituents of munitions systems to be recovered and re-used in military applications due to environmental protection.

Typical methods for extracting TNT from high explosives include melting out of and steaming out of the explosive from the projectile casing. These two methods are disadvantageous for being time consuming, inappropriate for mass reclamation of TNT, generating too much polluted waste water which is required to treat in prohibitively high cost, and being low efficiency.

U.S. Pat. No. 5,953,679 to Morris discloses a method for the extraction of TNT from a high explosive comprising contacting the high explosive with a supercritical fluid at a temperature above the melting temperature (e.g., 85 degrees Celsius) of TNT and at a pressure (e.g., 37.4 MPa) sufficient to extract the TNT. However, it is known that there are other explosive components in the high explosive. Thus, it is very possible that unexpected explosions may occur during the extraction. This is very dangerous.

It is also known that many explosive components have a very low solubility in carbon dioxide based supercritical fluid. Further, TNT has a very low solubility in carbon dioxide based supercritical fluid. Thus, a great volume of carbon dioxide based supercritical fluid is required for reclaiming TNT from the high explosive. This in turn adversely increases the cost.

Notwithstanding the prior art, the invention is neither taught nor rendered obvious thereby.

### SUMMARY OF THE INVENTION

It is therefore one object of the invention to provide a method for the retraction of an explosive component from a high explosive, comprising the steps of loading a high explosive containing an explosive component into a melting-out vessel; supplying a supercritical fluid to the melting-out vessel; and contacting the high explosive with the supercritical fluid at a temperature below the melting point of the explosive component and at a pressure sufficient to melt-out the explosive component.

In a first aspect of the invention, the explosive component is at least one of TNT and TNT-based high explosive.

In a second aspect of the invention, the TNT-based high explosive is selected from the group consisting of Comp B, Amatol, Octol, and Ammonal.

In a third aspect of the invention, the temperature is between about 50 and 75 degrees Celsius and the pressure are between about 15 and 40 MPa.

In a fourth aspect of the invention, the temperature is about 55 degrees Celsius and the pressure is about 25 MPa in optimum conditions.

In a fifth aspect of the invention, the explosive component melt-out from the warhead has at least 99%.

By utilizing the invention, the following advantages and benefits are obtained: Safety because the carbon dioxide as a supercritical fluid is employed at a temperature less than the melting point of TNT. It is an environmentally friendly method because no organic solvent is used for collection purpose. Carbon dioxide can be substantially completely recycled after the melt-out. Hence, no pollution is generated. Cost effectiveness because the method is time saving and batch based. The TNT melt-out is done in a melting state, resulting in an increase of the percentage of TNT being melted-out.

The above and other objects, features and advantages of the invention will become apparent from the following detailed description taken with the accompanying drawings.

### BRIEF DESCRIPTION OF THE DRAWINGS

FIG. 1 is a flow chart illustrating a view of a method for reclaiming high explosive from warhead by melting-out in supercritical fluid according to the invention;

FIG. 2 schematically shows a system used in carrying out the method of the invention;

FIG. 3 schematically shows the extraction vessel of FIG. 2 and a support for holding the extraction vessel; and

FIGS. 4A to 4F are photographs of an experiment for extracting TNT from a metal container served as warhead according to the invention;

### DETAILED DESCRIPTION OF THE INVENTION

Temperature and pressure at the critical-point are defined as the critical temperature (T_{C}) and critical pressure (P_{C}). The critical parameters for carbon dioxide are T_{C} first 31 degrees Celsius and P_{C} first 7.39 MPa. A supercritical fluid results when the temperatures and pressures of the materials are greater than their critical parameters. For effective melt-out of high explosive, operation is done at pressure of about 25 MPa and temperature of about 55 degrees Celsius. All high explosive melt-out were carried out using a carbon dioxide based supercritical fluid in a safe and cost effective manner because the liquid carbon dioxide is non-flammable, non-toxic, chemically stable, and cost effective.

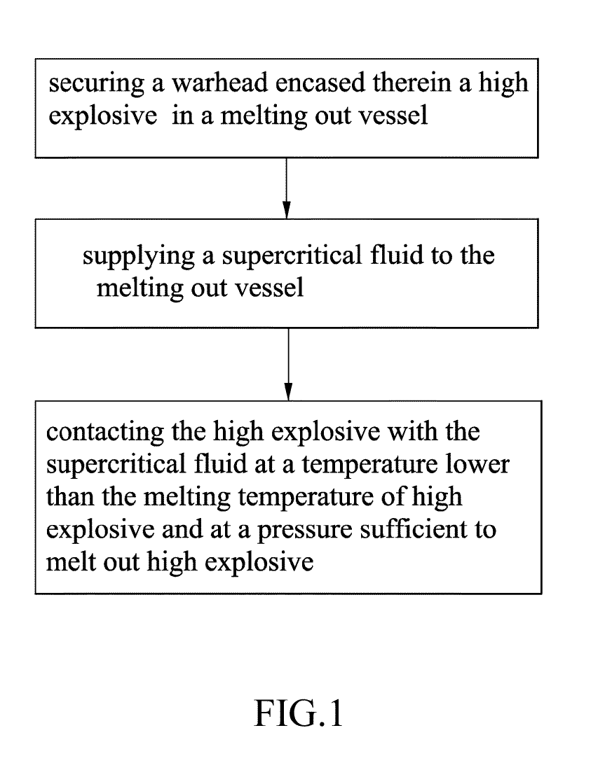

Referring to FIG. 1, a method for reclaiming high explosive from warhead by melting-out in supercritical fluid in accordance with the invention is illustrated. The method comprises the steps of securing a warhead having encased therein high explosive in a melt-out vessel; supplying a supercritical fluid to the melt-out vessel; and contacting the high explosive with the supercritical fluid at a temperature lower than the melting temperature of the high explosive and at a pressure sufficient to melt-out the high explosive.

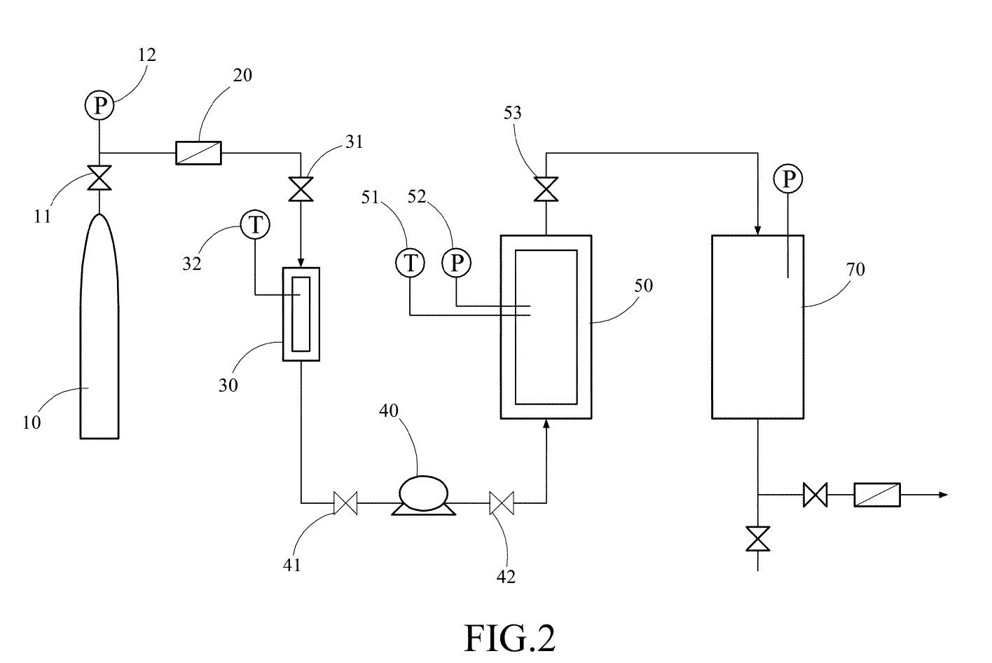

Referring to FIG. 2, a system for carrying out the method in accordance with the invention is schematically shown. In operation, liquid carbon dioxide is stored in a first storage tank 10. The liquid carbon dioxide is introduced to a filter 20 through a valve 11 by a pump 12. After being filtered by the filter 20, the pure liquid carbon dioxide passes a valve 31 into a second storage tank 30. The liquid carbon dioxide is heated to an operating temperature in the second storage tank 30 by a heater (not shown). The second storage tank 30 is provided with a thermometer 32 for measuring temperature of the liquid carbon dioxide contained therein. The heated liquid carbon dioxide is pressurized by a pump 40 prior to entering a melt-out vessel 50 in the state of a supercritical fluid. At upstream and downstream of the pump 40, there are provided valves 41, 42 respectively. The provision of the valves 41, 42 can adjust the amount of liquid carbon dioxide as supercritical fluid supplied to the melt-out vessel 50. The melt-out vessel 50 is provided with a thermometer 51 and a pressure gauge 52 so that a person operating the system may be visually aware of the temperature and the pressure of the liquid carbon dioxide contained in the melting-out vessel 50. Preferably, the temperature of the melt-out vessel 50 is sufficiently low and the pressure thereof is also sufficient so that the high explosive may contact the supercritical fluid at the predetermined temperature range and at the predetermined pressure range to melt-out the high explosive. The supercritical fluid may then flow to a flow restrictor 53 with the flow being reduced thereat. As the pressure drops, the liquid carbon dioxide becomes a gas at an ambient temperature, and any dissolved solute nucleates and is collected in a carbon dioxide recycling vessel 70. The expanded carbon dioxide gas flows to a subsequent station for further processing.

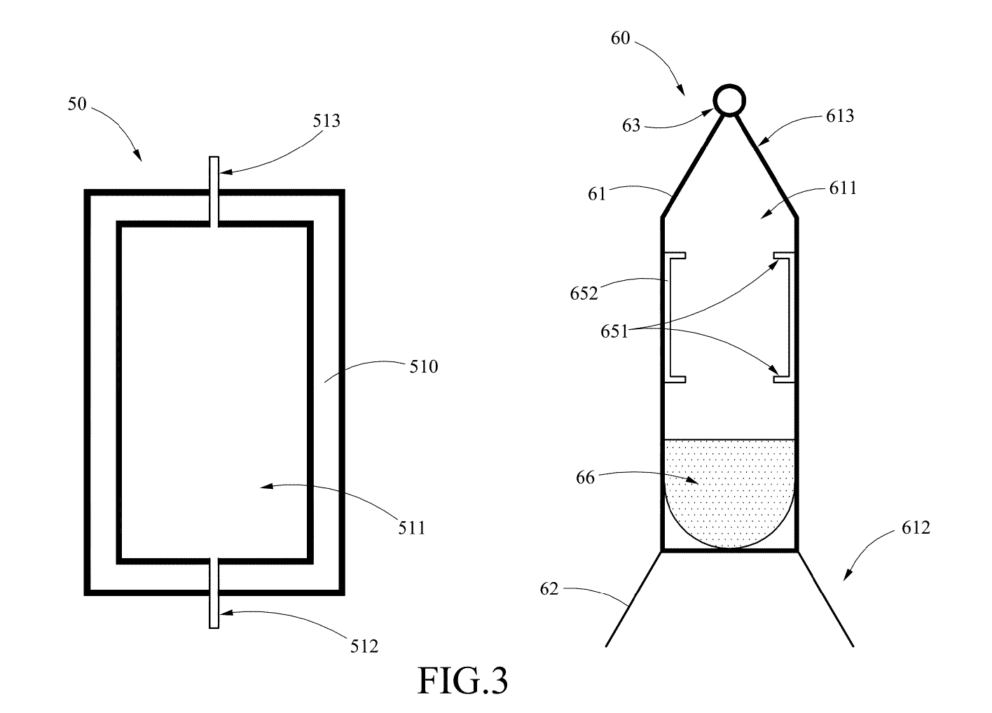

Referring to FIG. 3, the melt-out vessel 50 comprises a shell 510 with a temperature control layer (not shown), a space 511 defined by the shell 510, an inlet 512 through the bottom, and an outlet 513 through the top. A support 60 is provided for holding the melt-out vessel 50. The support 60 comprises a shell 61, a space 611 defined by the shell 61, an inlet 612 of the shell 61 communicating with the inlet 512 and being held by a support 62 which is placed in the melt-out vessel 50, an outlet 613 communicating with the outlet 513, a hanging ring 63 proximate the outlet 613 for moving the support 60, a pair of brackets 651, 652 for positioning a warhead (not shown), and a storage member 66 under the brackets 651, 652. In operation, high explosive of the warhead may contact the liquid carbon dioxide (i.e., supercritical fluid) at optimum temperature and pressure ranges. As a result, the high explosive is melted-out and temporarily stored in the storage member 66.

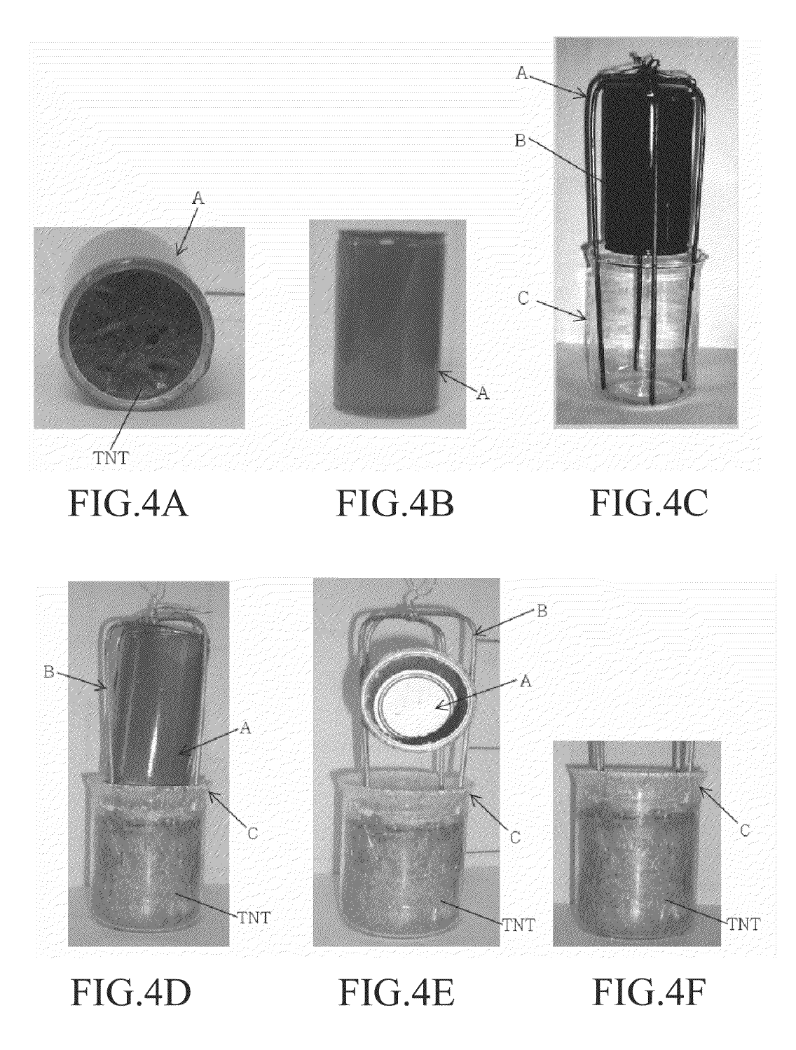

Referring to FIGS. 4A to 4F an experiment in accordance with the invention is conducted with the following equipment and conditions: A cylindrical metal container A (served as warhead) has a diameter of 60 mm, a bottom opening having a diameter of 50 mm, and a volume of 350 ml. TNT of 350 g is contained in the container A. support B is provided to hold the container A. A cup C is provided in the support B and is under the container A. Together they are disposed in an extraction vessel (not shown) having a volume of 2 liter prior to sealing (see FIGS. 4A to 4C).

In operation (see FIGS. 4D to 4F), pressurized liquid carbon dioxide as a supercritical fluid is supplied to the melt-out vessel which is heated to a temperature of about 55 degrees Celsius and at a pressure of about 25 MPa. TNT in the container A begins to melt when it contacts the supercritical fluid. It takes about 30 minutes to collect about 350 g of purity TNT in the cup C at the end of the operation. The collected TNT is cured in the cup C after cooling.

Following are examples of the invention with the space 511 having a volume of two (2) liters:

### Example (I) for Melting-Out TNT in Low Temperature

A simulated warhead of 40 mm diameter containing 60 g TNT in an inverted position is held by a support. Place both the support and the warhead in a melt-out vessel prior to sealing. TNT begins to melt from the warhead as pressurized liquid carbon dioxide as a supercritical fluid is supplied to the melt-out vessel. Percentages of the melted TNT are tabulated in the following Tables (I) and (II) as temperature and pressure vary in the melt-out operation which takes about 30 minutes.

TABLE (I)

| Pressure | Temperature | Time | % TNT |
|---|---|---|---|
| (MPa) | (degrees Celsius) | (min) | melted-out |
| 15 | 55 | 30 | 59.5 |
| 20 | | | 100 |
| 25 | | | 100 |
| 30 | | | 100 |
| 35 | | | 100 |
| 40 | | | 100 |

TABLE (II)

| Temperature | Pressure | Time | |
|---|---|---|---|
| (degrees Celsius) | (MPa) | (min) | % TNT melted-out |
| 35 | 25 | 30 | 2 |
| 45 | | | 5 |
| 55 | | | 100 |
| 65 | | | 100 |
| 75 | | | 100 |

Definition:
% TNT melt-out operation is defined by weight of the melted TNT divided by weight of TNT before the melt-out operation and multiplied by 100%.

### Example (II) for Melting-Out TNT in Low Temperature

A simulated warhead having a volume of 250 ml containing 250 g TNT in an inverted position is held by a support. Place both the support and the warhead in a melt-out vessel prior to sealing. TNT begins to melt-out from the warhead as pressurized liquid carbon dioxide as a supercritical fluid is supplied to the melt-out vessel which is maintained at a temperature of about 55 degrees Celsius and at a pressure of about 25 MPa. The melt-out operation takes about 30 minutes. Results: About zero (0) gram TNT is remained in the warhead and about 241 g of TNT is collected in the TNT collection vessel after the melt-out operation.

### Example (III) for Melting-Out TNT in Low Temperature

A simulated warhead having a volume of 350 ml containing 500 g TNT in an inverted position is held by a support. Place both the support and the warhead in a melt-out vessel prior to sealing. TNT begins to melt-out from the warhead as pressurized liquid carbon dioxide as a supercritical fluid is supplied to the melt-out vessel which is maintained at a temperature of about 55 degrees Celsius and at a pressure of about 25 MPa. The melt-out operation takes about 30 minutes. Results: About zero (0) gram TNT is remained in the warhead and about 490 g of TNT is collected in the TNT collection vessel after the melt-out operation.

### Example (IV) for Molting-Out Comp B in Low Temperature

A 105 mm howitzer warhead containing 2200 g composition B (i.e., Comp B) consisting of 60% RDX, 40% TNT, and less than 1% wax in an inverted position is held by a support. Place both the support and the warhead in a molt-out vessel prior to sealing. Composition B begins to molt-out from the warhead as pressurized liquid carbon dioxide as a supercritical fluid is supplied to the molt-out vessel which is maintained at a temperature of about 65 degrees Celsius and at a pressure of about 25 MPa. The melting-out operation takes about 120 minutes.

### Example (V) for Molting-Out TNT in Low Temperature

A 155 mm howitzer warhead containing 6700 g TNT in an inverted position is held by a support. Place both the support and the warhead in a molt-out vessel prior to sealing. TNT begins to molt-out from the warhead as pressurized liquid carbon dioxide as a supercritical fluid is supplied to the molt-out vessel which is maintained at a temperature of about 65 degrees Celsius and at a pressure of about 25 MPa. The melting-out operation takes about 30 minutes.

It is envisaged by the invention that the optimum temperature and pressure for melting-out TNT from a warhead are 55 degrees Celsius and 25 MPa respectively. The 55 degrees Celsius is less that the melting temperature of 80 degrees Celsius of TNT and the 25 MPa is less than the required pressure of 27.4 MPa respectively as compared with the conventional method of extracting TNT from a warhead. Hence, the invention is both safer and more cost effective as compared with the conventional TNT extraction method.

It is also envisaged by the invention that in the process of melting-out TNT from Comp B (as another high explosive) more than 99% TNT contained in the warhead can also be melted-out as mentioned above. Alternatively, the high explosive of the invention can be selected from the group consisting of Amatol, Octol, and Ammonal. While the invention has been described in terms of preferred embodiments, those skilled in the art will recognize that the invention can be practiced with modifications within the spirit and scope of the appended claims.

What is claimed is:

1. A method for the melt-out of an explosive component from a high explosive, comprising the steps of:
(a) loading a high explosive containing an explosive component into a melt-out vessel;
(b) supplying a supercritical fluid to the melt-out vessel; and
(c) contacting the high explosive with the supercritical fluid at a temperature below the melting point of the explosive component and at a pressure sufficient to melt-out the explosive component.

2. The method of claim 1, wherein the explosive component is at least one of TNT and TNT-based high explosive.

3. The method of claim 2, wherein the TNT-based high explosive is selected from the group consisting of Comp B, Amatol, Octol, and Ammonal.

4. The method of claim 2, wherein the supercritical fluid is liquid carbon dioxide.

5. The method of claim 2, wherein the temperature is between about 50 and 75 degrees Celsius and the pressure is between about 15 and 40 MPa.

6. The method of claim 5, wherein the temperature is about 55 degrees Celsius and the pressure is about 25 MPa.

7. The method of claim 3, wherein the explosive component melted-out from the TNT-based high explosive has at least 99% TNT.

8. The method of claim 1, wherein steps (a), (b), and (c) are done by batches operation.

9. The method of claim 2, wherein the TNT is melted by a melt-out method.

10. The method of claim 2, wherein the TNT-based high explosive is melted by a melt-out method.

11. The method of claim 1, wherein the temperature of the supercritical fluid is about 55 degrees Celsius and the pressure thereof is about 25 MPa.

12. The method of claim 2, wherein the temperature of the supercritical fluid is about 55 degrees Celsius and the pressure thereof is about 25 MPa.
